(19) United States
(12) Patent Application Publication
(10) Pub. No.: US 2012/0135489 A1
(43) Pub. Date: May 31, 2012

(54) METHOD OF ALCOHOL PRODUCTION

(75) Inventor: Karl Ragnar Weydahl, Bergen (NO)

(73) Assignee: WEYLAND AS, Blomsterdalen (NO)

(21) Appl. No.: 13/378,655

(22) PCT Filed: Jun. 8, 2010
(86) PCT No.: PCT/GB2010/001120

§ 371 Date: Feb. 21, 2012

(30) Foreign Application Priority Data
Jun. 19, 2009 (GB) 0910707.9

(51) Int. Cl. C12P 7/10; B01J 19/00; C13K 13/00

(52) U.S. Cl. 435/165; 127/37; 127/1

(57) ABSTRACT

The present invention relates to a process for producing alcohol from a cellulosic material, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; subjecting said residue to an oligosaccharide cleavage reaction to yield an aqueous solution of fermentable sugars; fermenting said fermentable sugars and distilling alcohol from the resulting fermented mixture; contacting said first aqueous acidic solution with a water-immiscible liquid lipophilic solvent to yield a second aqueous acid solution and a solvent mixture of said extraction solvent and said liquid solvent; separating said solvent mixture to yield extraction solvent for recycling; and separating from said second aqueous acid solution an aqueous acid for recycling.

The invention relates to improvements in and relating to a process for the production of alcohol, especially ethanol or butanol, from cellulosic materials, in particular a process involving acid hydrolysis of cellulose.

Alcohol, produced by fermenting biomass, is rapidly becoming a major alternative to hydrocarbons such as natural gas and petroleum. While the current focus is on the production of ethanol from plant seed, e.g. maize or sugar cane juice, the magnitude of the demand for alcohol threatens a reduction in the land area devoted to food production and a desirable alternative to plant seed as the starting material is plant material other than seed, e.g. grass, wood, paper, maize husks, straw, etc. In this case the ethanol is produced by first breaking down the cellulose and hemicellulose (for convenience both are simply referred to as cellulose herein) into fermentable sugars. This may be done with enzymes but it is achieved most efficiently and economically by hydrolysis with strong acids, for example mineral acids such as sulphuric and hydrochloric acid. However for large scale commercial production of alcohol in this way, a major portion of the acid used must be recovered and recycled.

In WO 02/02826, the contents of which are hereby incorporated by reference, the inventors proposed such an ethanol production process in which the strong acid was recovered by contacting the hydrolysate with an organic extraction solvent, for example methyl ethyl ketone, with separation of the solid lignin and precipitated sugars to yield an acid solution comprising water, extraction solvent, acid and some dissolved sugars. The extraction solvent in the acid solution was then evaporated off under vacuum to be recycled and to leave an aqueous acid and sugar solution which was further evaporated off to yield a concentrated acid/sugar mixture, again for recycling.

The hydrolysate:extraction solvent ratio used in WO 02/02826 (see Example 1) is of the order of 3:8 and accordingly the energy requirement for recovery of the extraction solvent for recycling is a major portion of the overall energy demand for converting the cellulosic raw material into distilled ethanol.

We have now found that the extraction solvent recovery may be effected efficiently and with significantly lower energy demand by contacting the acid solution with a water-immiscible liquid lipophilic solvent to generate two liquid product streams, one, the minor stream, a more concentrated aqueous acid solution and the other, the major stream, predominantly a mixture of the lipophilic solvent and the extraction solvent from the acid solution feed. Rectification of the major stream to yield an extraction solvent stream suitable for recycling is relatively less energy demanding than rectifying the acid solution feed itself, and rectification of the concentrated acid solution stream can be performed in more compact apparatus and/or in a batchwise fashion, again providing benefits in terms of energy and space demand. In this way, an energy saving of about 50% or more may be achieved in the extraction solvent recovery. Moreover, the water content of the recycled extraction solvent may be significantly reduced.

Thus viewed from one aspect the invention provides a process for producing alcohol from a cellulosic material, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; subjecting said residue to an oligosaccharide cleavage reaction to yield an aqueous solution of fermentable sugars; fermenting said fermentable sugars and distilling alcohol from the resulting fermented mixture; contacting said first aqueous acidic solution with a water-immiscible, e.g. water-insoluble liquid lipophilic solvent to yield a second aqueous acid solution and a solvent mixture of said extraction solvent and said liquid solvent; separating said solvent mixture to yield extraction solvent for recycling; and separating from said second aqueous acid solution an aqueous acid for recycling.

The lipophilic solvent is preferably a halocarbon, or a hydrocarbon (e.g. an alkane, alkene, alkyne, or a low-boiling aromatic hydrocarbon such as for example benzene, toluene or xylene), or mixture thereof. The halocarbon or hydrocarbons used will conveniently have a carbon content of up to 8 atoms, e.g. 1 to 6 atoms, especially 5 atoms. Particularly desirably the solvent is a combustible material suitable for combustion to provide energy for one or more steps in the overall process. Especially preferably it is a material that is commercially available in liquid or liquefied form, particularly a hydrocarbon or hydrocarbon mixture. Accordingly, the lipophilic solvent is desirably hexane or a hexane mixture, pentane or a pentane mixture, butane or a butane mixture, propane, ethane or a liquefied hydrocarbon gas, e.g. liquefied petroleum gas (LPG) or liquefied natural gas. While liquefied gases may be flashed off from the major stream by depressurisation, their subsequent recycling requires liquefaction and thus is energy demanding. Accordingly if liquefied gases are used, they will generally be combusted to provide energy for one or more steps of the process rather than being liquefied and recycled. They moreover require pressurised storage vessels and they require the separation column to be pressure resistant. As a result, the use of lipophilic solvents which are liquid at ambient conditions (e.g. 20° C. and 1 atmosphere) is preferred. The use of pentane mixtures is preferred. Pentanes are especially preferred as they do not form azeotropic mixtures with the extraction solvents normally usable.

The boiling points of the extraction solvent and the lipophilic solvent at 1 atmosphere are preferably separated by at least 10° C., especially at least 20° C., more especially at least 30° C., so as to facilitate their separation. The extraction solvent is conveniently the higher boiling of the two.

The lipophilic solvent will preferably be contacted with the first aqueous acidic solution at a temperature between 0 and 80° C., especially 10 and 60° C., more particularly 15 and 50° C. The pressure used will be one sufficient to maintain the lipophilic solvent in liquid form at the contact temperature used. If not already known, such pressures may readily be determined experimentally.

The contact between the lipophilic solvent and the water/acid/extraction solvent is preferably effected in a counterflow separation column with the lipophilic solvent being fed in at the base, the water/acid/extraction solvent being fed in at the top, the major extraction solvent/lipophilic solvent stream being discharged from the top and the minor water/acid stream being discharged from the base. The separation column is preferably equipped with static or active mixers and/or deflection plates so as to ensure thorough mixing.

The weight ratio of the inflowing acidic solution and lipophilic solvent feeds is preferably in the range 7:1 to 1:1, especially 5:1 to 2:1, particularly 4:1 to 3:1. The weight ratio of the discharged major and minor streams is preferably similar, e.g. also in the range 7:1 to 1:1, especially 5:1 to 2:1, particularly 4:1 to 3:1.

The outflowing major stream is preferably separated on a continuous basis, e.g. by temperature increase and/or pressure decrease. Particularly preferably the pressure used is one at which the lipophilic solvent will recondense if cooled using ambient water, e.g. at 4-25° C. The resultant extraction solvent stream (the bottom product) will generally be sufficiently pure for recycling to the separation column in which hydrolysate and extraction solvent are contacted. The resultant lipophilic solvent stream (the top product) will also generally be sufficiently pure for recycling or combustion.

The outflowing aqueous acidic solution stream may be separated on a continuous basis, or more preferably batchwise. This can be done by distillation at elevated temperature and/or reduced pressure, generally elevated temperature. The resultant aqueous acid stream (the bottom product) may be recycled to the hydrolysis reactor, optionally after concentration. The resultant extraction solvent stream (the top product) may be combusted or recycled to the separation column where hydrolysate is contacted with extraction solvent.

The overall alcohol production process may if desired be performed at a set of production sites, e.g. with production of the fermentable sugars on one site and fermentation and distillation at another. Equally, the acid hydrolysis, acid removal and extraction solvent removal may be performed at one site with the oligosaccharide cleavage and other downstream steps being performed at another site. Thus viewed from a further aspect the invention provides a process for the production of an aqueous solution of fermentable sugars from a cellulosic material, which process comprises: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; contacting said first aqueous acidic solution with a water-immiscible, e.g. water-insoluble liquid lipophilic solvent to yield a second aqueous acid solution and a solvent mixture of said extraction solvent and said liquid solvent; separating said solvent mixture to yield extraction solvent for recycling; and separating from said second aqueous acid solution an aqueous acid for recycling.

Viewed from another aspect the invention provides a process for the production of a sugar composition, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; drying said residue to yield said sugar composition; contacting said first aqueous acidic solution with a water-immiscible, e.g. water-insoluble liquid lipophilic solvent to yield a second aqueous acid solution and a solvent mixture of said extraction solvent and said liquid solvent; separating said solvent mixture to yield extraction solvent for recycling; and separating from said second aqueous acid solution an aqueous acid for recycling.

The acid used in the process of the invention may be any strong acid, but will generally be an inorganic acid such as phosphoric or sulphuric acid. The use of sulphuric acid is preferred; the use of hydrochloric acid is generally not preferred. The use of a mixture of sulphuric and phosphoric acids, e.g. in a 1:1 to 4:1 volume ratio, especially about 2:1 volume ratio, is especially preferred.

The acid solution as contacted with the cellulosic starting material preferably corresponds to an acid:water weight ratio of 1:1 to 4:1, especially about 3:1. Acid solutions of the acid strengths conventionally used in strong acid hydrolysis of cellulosic materials may be used. It should be noted that acid and water may be added separately or that the initial acid added may be diluted or concentrated to yield the desired acid:water balance.

The acid hydrolysis may be performed in conventional fashion. Typically, hydrolysis, which is exothermic, will be performed on a continuous basis, under cooling, e.g. water cooling, to maintain the hydrolysis mixture at 50 to 55° C. The acid solution:cellulosic material ratio is typically 2:1 to 4:1 by weight and the hydrolysis duration will generally be 1 to 4, especially about 2, hours. In this way the cellulose is broken down to produce oligosaccharides which can be precipitated out by the extraction solvent to yield a lignin/sugars slurry.

The extraction solvent used in the process of the invention may be any organic solvent which is capable of taking up water and acid and thereby causing the sugars to precipitate. Typically the solvent will be an alcohol, ether or ketone, e.g. having up to eight carbons. A mixture of such solvents, e.g. as described in WO 02/02826 may of course be used. The use of methyl ethyl ketone is preferred.

Contact between hydrolysate and extraction solvent is preferably effected in a counter flow column such that extraction solvent is added from below and removed from above and hydrolysate is added from above and the lignin/sugars slurry is removed from below. The slurry may be washed with extraction solvent if desired, it may be drained of liquids if desired, and it may be dried if desired. Alternatively it can be used directly for the oligosaccharide cleavage step after addition of water to bring the sugars into solution. The oligosaccharide cleavage reaction may be effected enzymatically or alternatively, and preferably, by acid hydrolysis. In practice the residue of acid retained in the unwashed slurry is adequate for oligosaccharide cleavage to proceed via such a second acid hydrolysis step. Alternatively further acid may be added, for example to bring the acid content of the sugar solution up to about 0.1 to 5 wt %, especially 0.5 to 2 wt %, particularly about 1 wt %. Addition of excess acid is undesirable as, following a second acid hydrolysis, the resulting hydrolysate must be neutralized to a pH suitable for the microorganisms responsible for fermentation (generally yeasts). This second hydrolysis may be effected under conventional conditions for weak acid hydrolysis of oligosaccharides, e.g. a temperature of 125 to 155° C., particularly about 140° C., a pressure of 2 to 7 bar, preferably 5-6 bar, and a duration of about two hours.

Before fermentation, the fermentable sugars in aqueous solution are preferably filtered to recover any lignin. This is preferably washed to recover any entrained sugars for fermentation and compressed for use as a fuel, e.g. to provide energy for one or more of the steps in the overall alcohol production process.

Where the raw cellulosic material is rice straw. the lignin/sugars mixture will contain fine silica particles. These may be recovered by filtration, e.g. using differently sized meshes for lignin and silica or they may be recovered from the residue of the combustion of the lignin. Such silica particles are useful, e.g. as paint additives, pharmaceutical tabletting aids, or catalyst carriers (e.g. for olefin polymerization), and their collection and use form further aspects of the present invention.

The microorganism used in the fermentation step may be any microorganism capable of converting fermentable sugars to alcohol, e.g. brewer's yeast. Preferably however a yeast or yeast mixture is used which can transform the pentoses yielded by hemicellulose hydrolysis as well as the hexoses yielded by cellulose hydrolysis. Such yeasts are available commercially. The use of microorganisms that can transform pentoses to alcohol (e.g. Pichia stipitis, particularly P. stipitis CBS6054), particularly in combination with ones which can transform hexoses to alcohol, is especially preferred. Where fermentation is performed using microorganisms other than brewer's yeast (e.g. C. beijerinckii BA101), alcohols other than ethanol, in particular butanol, can be produced and these too can be used as biofuels. The invention covers the production of such other alcohols.

Distillation may be effected in conventional fashion.

The sugars produced using the invention can be fermented or respired by Baker's yeast or other microorganisms yeast to yield many different biological produced compounds such as glycerol, acetone, organic acids (e.g. butyric acid, lactic acid, acetic acid), hydrogen, methane, biopolymers, single cell protein (SCP), antibiotics and other pharmaceuticals. Specific proteins, enzymes or other compounds could also be extracted from cells grown on the sugars. The sugars moreover may be transformed into desired end products by chemical and physical rather than biological means, e.g. reflux boiling of xylose will yield furfural. The invention thus also covers the production of all such other produced compounds besides alcohols.

Viewed from another aspect, the invention provides apparatus for use in the processes of the invention, said apparatus comprising: a hydrolysis reactor; a first separator arranged to receive hydrolysate from said reactor and to discharge a sugar slurry; a second separator arranged to receive an extraction solvent/water mixture from said first separator and to discharge (a) an aqueous acid solution and (b) an extraction solvent/lipophilic solvent mixture; an acid reservoir arranged to supply acid to said reactor; an extraction solvent reservoir arranged to supply an organic extraction solvent to said first separator; a lipophilic solvent reservoir arranged to supply a water-immiscible liquid lipophilic solvent to said second separator; a first rectifier arranged to receive an extraction solvent/lipophilic solvent mixture from said second separator and to discharge (a) lipophilic solvent and (b) extraction solvent; and a second rectifier arranged to receive a aqueous acid solution from said second separator and to discharge (a) a concentrated aqueous acid and (b) extraction solvent; and recycling conduits arranged to return extraction solvent to said first separator or an extraction solvent reservoir and to return concentrated aqueous acid to said reactor or an acid reservoir.

The apparatus preferably also comprises components for feeding cellulosic material to the reactor. Conveniently, it also comprises components for the downstream handling of the sugar slurry, e.g. further hydrolysis reactors, reservoirs for a base for neutralizing the residual acid, fermentors and distillation units. To allow for continuous operation of the process when individual steps are performed batchwise, individual units within the apparatus may be duplicated, i.e. with such units being in parallel, so that one may be in operation while the other is being loaded/unloaded. This is particularly the case for the second acid hydrolysis, the fermentation, the distillation, and the lignin separation steps.

Where fermentation is performed using microorganisms other than brewer's yeast (e.g. C. beijerinckii BA101), alcohols other than ethanol, in particular butanol, can be produced and these too can be used as biofuels. The invention covers the production of such other alcohols.

Embodiments of the invention will now be described further with reference to the following non-limiting Examples and the accompanying drawing, in which:

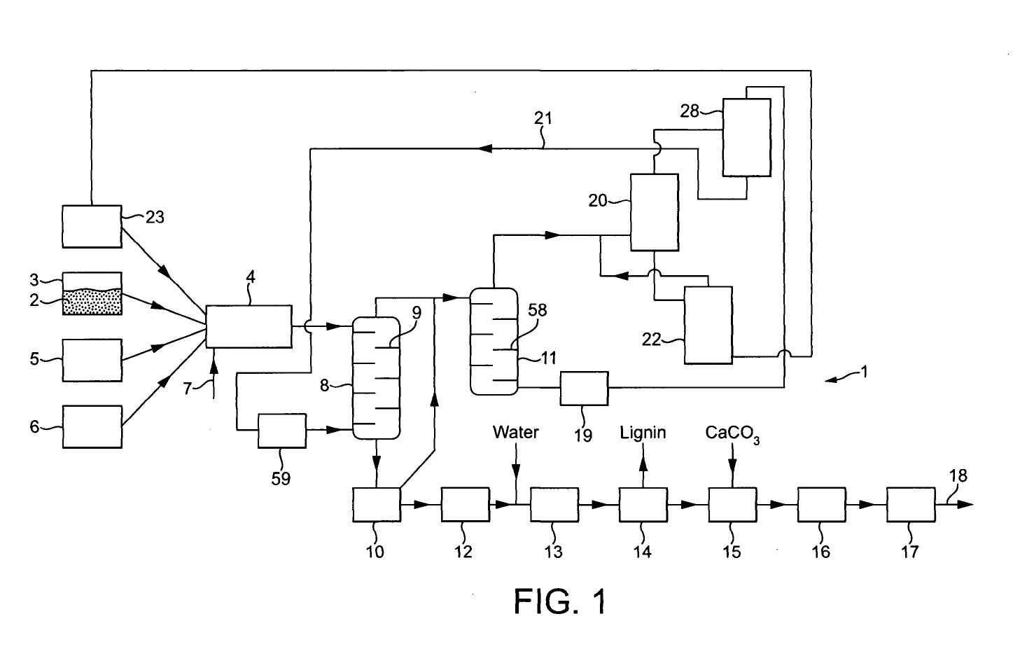

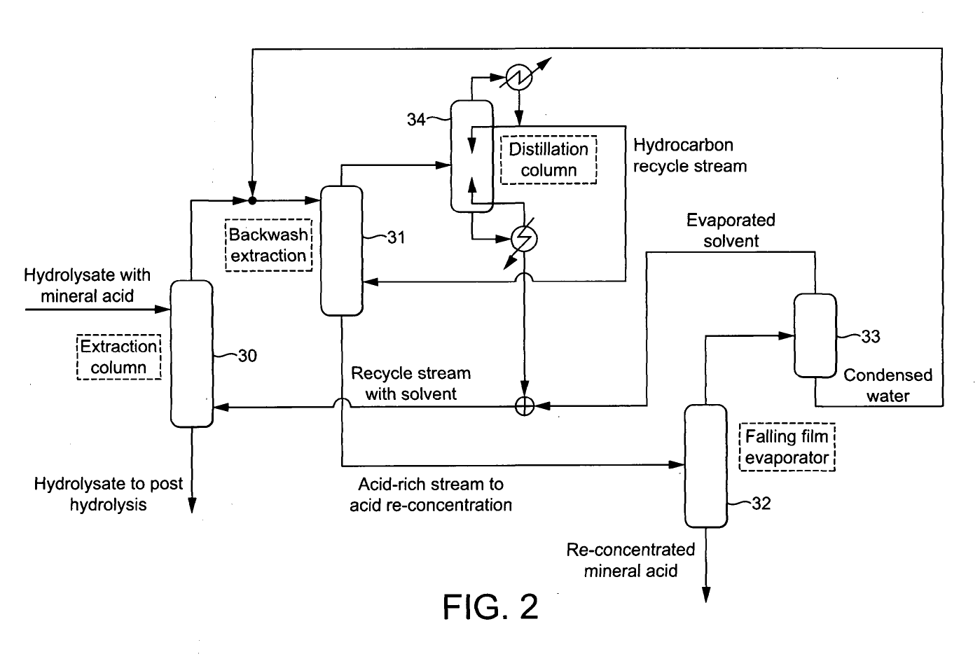

FIGS. 1 and 2 are schematic diagrams of apparatus according to the invention.

Referring to FIG. 1, there is shown an apparatus 1 for the conversion of wood pulp to ethanol. Wood pulp 2 is fed from hopper 3 into a hydrolysis reactor 4 containing a rotating screw operated to ensure a residence time for the wood pulp within the reactor of about two hours. The reactor is provided with a water-cooling jacket to maintain the hydrolysis mixture at about 50-55° C. Sulphuric and phosphoric acids and water, in a weight ratio of 2:1:1 are fed into reactor 4 from reservoirs 5 and 6, water feed line 7, and acid recycling reservoir 23. The hydrolysate from reactor 4 is fed to the top of a counterflow separation column 8 having internal plates 9 to delay through flow. Into the base of column 8 is introduced an organic extraction solvent, methyl ethyl ketone (MEK) from reservoir 59. Within the column 8, water and acid are taken up by the extraction solvent and lignin and precipitated sugars are passed from the base of the column to a continuous filtration unit 10. The acid/water/extraction solvent mixture is discharged from the top of column 8 and fed into a separator column 11 also equipped with plates 58.

The solid residue from filtration unit 10 is passed to a drier 12 and the dry lignin/sugar mixture is then dissolved in water and passed into a second hydrolysis reactor 13. The liquid from the filtration unit 10 is passed to separator column 11.

In the second reactor 13, a further acid hydrolysis is effected at 140° C. for two hours at 5-6 bar. The hydrolysate is filtered in filtration unit 14 to remove lignin (which is compressed and combusted to provide energy for the overall apparatus). The remaining solution of fermentable sugars is neutralized with calcium carbonate in neutralization unit 15 before being passed to fermentation unit 16 where brewers' yeast is added and fermentation is allowed to take place. The fermented mixture is then fed to distillation unit 17 where ethanol is distilled off via line 18.

The acid/water/extraction solvent is contacted in counterflow manner in separator column 11 with a pentane mixture from pentane reservoir 19. The resultant pentane/MEK major stream is led from separator column 11 to a rectifier 20 where the temperature is increased sufficiently to distill of pentane and extraction solvent as a gas which is fed to rectifier 28 where pentane is distilled off and recycled to reservoir 19. The MEK is recycled via line 21 to reservoir 59. The aqueous acid from rectifier 20 is fed to rectifier 22 where MEK is distilled off and recycled into rectifier 20. By operating rectifier 22 batchwise, the MEK can be distilled off first and then water may be distilled off to leave a concentrated acid. The remaining acid, containing some dissolved sugar, is recycled to reservoir 23.

Referring to FIG. 2, it can be seen how the extraction solvent from the extraction column 30 is separated from the acid in the “backwash extraction column” 31 by the hydrocarbon. The acid is then concentrated up before recycling to the hydrolysis reactor (not shown) in a falling film evaporator 32, with the top product being passed to a distillation column 33 to yield an extraction solvent stream which is recycled into the extraction column and an aqueous stream which is recycled into the backwash extraction column. The hydrocarbon from the backwash extraction column, containing most of the extraction solvent then passes to a distillation column 34 where the two are separated, again for recycling.

The appropriate number of theoretical stages in the extraction columns may be determined conventionally from a McCabe-Thiele diagram.

FIGS. 1 and 2 both show a single extraction column for stripping extraction solvent using hydrocarbon. Multiple columns, in series, may of course be used if desired.

### EXAMPLE 1

Acid Hydrolysis, Sugar Recovery and Fermentation

Sulphuric acid 36.2 g (38.1 g, 95% commercially available acid) was added to a batch of recirculated diluted acid solution containing 150.0 g phosphoric acid and 263.8 g sulphuric acid. The acid concentration in the solution was increased to 58.9% by weight by vacuum evaporation.

The concentrated acid solution was mixed with 150.0 g bagasse and warmed at 50° C. with mechanical stirring for 2.0 hrs.

The resultant slurry, containing hydrolyzed cellulose and solid lignin, was cooled to ambient temperature and mixed with an extraction solvent consisting of methanol (4.5% by weight), isopropanol (4.4% by weight), 2-butanone (85.3% by weight), and water (5.8% by weight).

The mixture of solid particles (lignin, precipitated sugars and residual acid) and solvent was separated into a solid phase and an extract phase, the latter containing the bulk of the total mineral acid (85.8% of total acid). The concentration of mineral acid in the extract was 14.2% by weight.

Subsequent washing of the solid phase containing precipitated sugars further reduced the acid content in the solid phase. If desired, the wash solution recovered could be added to the extract phase, however this was not done in this experiment.

The solid phase was suspended in water and residual extraction solvent was boiled off under vacuum. The suspension, containing dissolved sugars and solid lignin was warmed at 140° C. in an autoclave for 1.25 hrs. After cooling, the un-dissolved lignin and the sugar solution were separated by filtration. The acidity of the sugar-containing filtrate was adjusted to pH=4.5 with calcium carbonate. Precipitated calcium sulphate was then separated from the sugar solution by filtration. The filter cake was washed with water to reclaim any sugars trapped in the filter cake.

A 100.0 mL sample of the sugar solution was diluted to 0.3 L with water. Bakers Yeast, 0.5 g, was added to the solution and the sugar solution was fermented at 30° C. to yield ethanol. After fermentation the solution was analysed with gas chromatography. The ethanol yield was calculated to be 1.44 mL which corresponds to 20.3 g/L of fermentable sugars prior to fermentation.

Combining sugar solutions from 5 subsequent experiments, each from hydrolyzing 150.0 g bagasse, gave an ethanol yield of 0.103 mL/g bagasse.

### EXAMPLE 2

Separation of Extraction Solvent from the Extract Phase (I)

Water, 10.0 g, and cyclohexane, 40.0 g, were added to a 99.9 g sample of the extract phase from Example 1. After mixing by shaking, the mixture was separated into two liquid phases. Samples of the organic phase with solvent, 95.1 g, and the lower acid-rich aqueous phase, 53.2 g, were neutralized with calcium carbonate and analyzed with gas chromatography. The acid concentrations in each phase were determined with acid-base titration. The water concentration in the organic phase was determined with Karl Fischer titration.

The calculated solvent recovery into the organic phase was 57.0 g, which was 73.6% by weight of total organics in the extract phase sample.

The water concentration in the organic phase was 1.0% by weight and the acid concentration was 0.17% by weight (3.3% of the total acid in the extract phase sample).

The acid content of the acid-rich phase was 13.11 g (24.7% by weight), the organic content of the acid-rich phase was 20.4 g (38.3% by weight) and the combined content of water and residual sugars was 19.7 g (37.0% by weight).

### EXAMPLE 3

Separation of Extraction Solvent from the Extract Phase (II)

The acid-rich phase from Example 2, 50.29 g, was mixed with cyclohexane, 39.88 g. The mixture was separated into an organic phase containing the bulk of residual solvent and an acid-rich aqueous phase.

Samples of the organic phase, 45.51 g, and of the acid-rich aqueous phase, 41.98 g, were neutralized with calcium carbonate and analyzed with gas chromatography.

The acid concentrations in each phase were determined with acid-base titration. The water concentration in the organic phase was determined with Karl Fischer titration.

The calculated fraction of solvent recovery into the organic phase was 7.98 g, which is 41.4% by weight of total organics in the acid-rich aqueous phase from Example 2. The water concentration in the organic phase was 0.18% by weight and the acid concentration 0.05% by weight (0.55% of the total acid in the acid-rich phase from Example 2).

The acid content of the acid-rich phase was 12.3 g (29.2% by weight), the organic content of the acid-rich was 10.8 g (25.7% by weight) and the combined content of water and residual sugars was 18.9 g (45.1% by weight).

### EXAMPLE 4

Separation of Extraction Solvent from the Extract Phase (III)

The acid-rich phase from Example 3, 39.42 g, was mixed with cyclohexane, 40.10 g. The mixture was separated into an organic phase containing the bulk of residual solvent and an acid-rich aqueous phase.

Samples of the organic phase, 41.52 g, and the acid-rich phase, 36.27 g, were neutralized with calcium carbonate and analyzed with gas chromatography. The acid concentrations in each phase were determined with acid-base titration. The water concentration in the organic phase was determined with Karl Fischer titration.

The calculated fraction of solvent recovery into the organic phase was 2.98 g, which is 29.4% by weight of total organics in the acid-rich phase from Example 3. The water concentration in the organic phase was approximately 0% by weight and the acid concentration 0.02% by weight (0.17% of total acid in the acid-rich phase from Example 2).

The acid content of the acid-rich phase was 11.1 g (30.7% by weight), the organic content of the acid-rich phase was 6.0 g (14.1% by weight) and the combined content of water and residual sugars was 20.2 g (55.2% by weight).

### EXAMPLE 5

Acid Hydrolysis, Sugar Recovery and Fermentation

Diluted recirculated mineral acid containing 300 g acid (about 180 g sulphuric acid and 120 g phosphoric acid) was reconcentrated to 72.4% by weight mineral acid. The concentrated acid was mixed with bagasse, 100.0 g, and warmed at 50° C. under mechanical stirring for 2 hrs. The resultant slurry, containing hydrolyzed cellulose and solid lignin, was cooled to ambient temperature and mixed with an extraction solvent consisting of isopropanol (15% by volume), 2-butanone (80.0% by volume), and n-pentane (5.0% by volume).

The resultant mixture of solid particles (lignin, precipitated sugars and residual acid) and solvent was separated into a solid phase, and an extract phase containing the bulk of the total mineral acid (88.6% by weight of total acid). The acid concentration in the extract phase was 1.28 M (ca 14% by weight).

Subsequent washing of the solid phase containing precipitated sugars further reduced the acid content in the solid phase.

The solid phase was suspended in water and residual extraction solvent was boiled off under vacuum. The remaining suspension containing dissolved sugars and solid lignin was warmed at 140° C. in an autoclave for 2.0 hrs. After cooling, the un-dissolved lignin and the sugar solution were separated by filtration. The acidity of the filtrate was adjusted to pH=4.5 with calcium carbonate.

Precipitated calcium sulphate was separated from the sugar solution by filtration. The filter cake was washed with water to reclaim any sugars trapped in the filter cake.

Bakers Yeast, 1.0 g, was added to the combined filtrate, and the sugars were fermented at 30° C. to yield ethanol. After fermentation, the solution was analysed with gas chromatography. The ethanol yield was calculated to be 13.72 mL.

### EXAMPLE 6

Separation of Extraction Solvent from the Extract Phase (I)

Methanol, 3.89 g, and n-pentane, 30.6 g, were added to a 90.4 g sample of the extract phase from Example 5. After mixing by shaking, the mixture was separated into two liquid phases. Samples of the organic phase with extraction solvent, 66.8 g, and of the acid-rich aqueous phase, 52.8 g, were neutralized with calcium carbonate and analyzed with gas chromatography.

The calculated fraction of solvent recovery into the organic phase was 41.8 g, which is 52.2% by weight of total organics.

The acid concentrations in each phase were determined with acid-base titration.

The acid concentration in the organic phase was 1.7% by weight (8.9% of total acid in the extract phase sample).

The acid content of the acid-rich phase was 11.3 g (21.3% by weight) and the organic content of the acid-rich phase was 38.8 g (73.0% by weight).

### EXAMPLE 7

Separation of Extraction Solvent from the Extract Phase (II)

The acid-rich phase from Example 6 was mixed with the organic phase from Example 6. The total weight of the mixture was 116.1 g. Water, 4.8 g, was added to the mixture and the mixture was shaken vigorously. The mixture was then allowed to settle into an acid-rich phase and an organic phase.

Samples of the organic phase with extraction solvent, 58.5 g, and of the lower acid-rich phase, 57.4 g, were neutralized with calcium carbonate and analyzed with gas chromatography.

The calculated fraction of solvent recovery into the organic phase was 41.8 g, which is 48.6% by weight of total organics.

The acid concentrations in each phase were determined with acid-base titration.

The acid concentration in the organic phase was 0.9% by weight (4.5% of the total acid in the extract phase sample). The water concentration in the organic phase was close to 0% by weight (analyzed with Karl Fisher titrations).

The acid content of the acid-rich phase was 11.5 g (20.0% by weight) and the organic content of the acid-rich phase was 40.5 g (70.0% by weight).

### EXAMPLE 8

Separation of Extraction Solvent from the Extract Phase (III)

The acid-rich phase from Example 7, 56.0 g, was mixed with n-pentane, 30.8 g. The mixture was then allowed to settle into an acid-rich phase and an organic phase.

Samples of the organic phase with extraction solvent, 43.9 g, and of the acid-rich phase, 41.5 g, were neutralized with calcium carbonate and analyzed with gas chromatography.

The calculated fraction of solvent recovery into the organic phase was 19.4 g, which is 48.0% by weight of the organics in the acid-rich phase from Example 7.

The acid concentrations in each phase were determined with acid-base titration.

The acid concentration in the organic phase was 0.1% by weight (0.3% of the total acid in the extract phase sample). The water concentration in the organic phase was close to 0% by weight (analyzed with Karl Fisher titrations).

The acid content of the acid-rich phase was 10.9 g (26.3% by weight) and the organic content of the acid-rich phase was 21.0 g (50.0% by weight).

### EXAMPLE 9

Separation of Extraction Solvent from the Extract Phase (IV)

The acid-rich phase from Example 8, 40.3 g, was mixed with n-pentane, 30.4 g. The mixture was then allowed to settle into an acid-rich phase and an organic phase.

Samples of the organic phase with extraction solvent, 34.1 g, and of the acid-rich phase, 35.0 g, were neutralized with calcium carbonate and analyzed with gas chromatography.

The calculated fraction of solvent recovery into the organic phase was 5.3 g, which is 24.1% by weight of organics in the acid-rich phase from Example 8.

The acid concentrations in each phase were determined with acid-base titration.

The acid concentration in the organic phase was 1.5% by weight (4.6% of the total acid in the extract phase sample). The water concentration in the organic phase was 0.7% by weight (analyzed with Karl Fisher titrations).

The acid content of the acid-rich phase was 10.5 g (30.0% by weight) and the organic content of the acid-rich phase was 16.6 g (47.0% by weight).

1. A process for producing alcohol from a cellulosic material, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; subjecting said residue to an oligosaccharide cleavage reaction to yield an aqueous solution of fermentable sugars; fermenting said fermentable sugars and distilling alcohol from the resulting fermented mixture; contacting said first aqueous acidic solution with a water-immiscible liquid lipophilic solvent to yield a second aqueous acid solution and a solvent mixture of said extraction solvent and said liquid solvent; separating said solvent mixture to yield extraction solvent for recycling; and separating from said second aqueous acid solution an aqueous acid for recycling.

2. A process for the production of an aqueous solution of fermentable sugars from a cellulosic material or a sugar composition, which process comprises: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; optionally drying said residue to yield said sugar composition; contacting said first aqueous acidic solution with a water-immiscible liquid lipophilic solvent to yield a second aqueous acid solution and a solvent mixture of said extraction solvent and said liquid solvent; separating said solvent mixture to yield extraction solvent for recycling; and separating from said second aqueous acid solution an aqueous acid for recycling.

3. (canceled)

4. A process as claimed in claim 1 or 2, wherein said second aqueous acid solution is separated by distillation to yield said aqueous acid for recycling.

5. A process as claimed in claim 1 or 2, wherein said lipophilic solvent comprises a halocarbon or hydrocarbon having up to eight carbon atoms.

6. A process as claimed in claim 5, wherein said lipophilic solvent is a pentane mixture.

7. A process as claimed in claim 1 or 2 wherein the boiling points of said extraction solvent and said lipophilic solvent at 1 atm are separated by at least 10° C., preferably at least 20° C.

8. The process as claimed in claim 1 or 2 wherein at 1 atm said extraction solvent is higher boiling than said lipophilic solvent.

9. Apparatus for use in a process as claimed in claim 1 or 2, said apparatus comprising: a hydrolysis reactor; a first separator arranged to receive hydrolysate from said reactor and to discharge a sugar slurry; a second separator arranged to receive an extraction solvent/water mixture from said first separator and to discharge (a) an aqueous acid solution and (b) an extraction solvent/lipophilic solvent mixture; an acid reservoir arranged to supply acid to said reactor; an extraction solvent reservoir arranged to supply an organic extraction solvent to said first separator; a lipophilic solvent reservoir arranged to supply a water-immiscible liquid lipophilic solvent to said second separator; a first rectifier arranged to receive an extraction solvent/lipophilic solvent mixture from said second separator and to discharge (a) lipophilic solvent and (b) extraction solvent; and a second rectifier arranged to receive a aqueous acid solution from said second separator and to discharge (a) a concentrated aqueous acid and (b) extraction solvent; and recycling conduits arranged to return extraction solvent to said first separator or an extraction solvent reservoir and to return concentrated aqueous acid to said reactor or an acid reservoir.

10. A process as claimed in claim 4, wherein said lipophilic solvent comprises a halocarbon or hydrocarbon having up to eight carbon atoms.

11. A process as claimed in claim 10, wherein said lipophilic solvent is a pentane mixture.

12. A process as claimed in claim 4 wherein the boiling points of said extraction solvent and said lipophilic solvent at 1 atm are separated by at least 10° C., preferably at least 20° C.

13. A process as claimed in claim 5 wherein the boiling points of said extraction solvent and said lipophilic solvent at 1 atm are separated by at least 10° C., preferably at least 20° C.

14. A process as claimed in claim 6 wherein the boiling points of said extraction solvent and said lipophilic solvent at 1 atm are separated by at least 10° C., preferably at least 20° C.

15. The process as claimed in claim 4 wherein at 1 atm said extraction solvent is higher boiling than said lipophilic solvent.

16. The process as claimed in claim 5 wherein at 1 atm said extraction solvent is higher boiling than said lipophilic solvent.

17. The process as claimed in claim 6 wherein at 1 atm said extraction solvent is higher boiling than said lipophilic solvent.

18. The process as claimed in claim 7 wherein at 1 atm said extraction solvent is higher boiling than said lipophilic solvent.
